(19) United States
(12) Patent Application Publication
(10) Pub. No.: US 2010/0285125 A1
(43) Pub. Date: Nov. 11, 2010

(54) DELIVERY SYSTEM FOR POORLY SOLUBLE DRUGS

(76) Inventors: Padma Venkitachalam Devarajan, Mumbai (IN); Joshi Vishvesh Mahendrakumar, Mumbai (IN); Krishna Vishnupad, Dayton, OH (US)

Correspondence Address: SEYFARTH SHAW LLP, WORLD TRADE CENTER EAST, TWO SEAPORT LANE, SUITE 300, BOSTON, MA, 02210-2028, US

(21) Appl. No.: 12/775,763

(22) Filed: May 7, 2010

Related U.S. Application Data
(60) Provisional application No. 61/176,256, filed on May 7, 2009.

(51) Int. Cl. A61K 9/24; A61K 9/14; A61P 9/12; A61P 3/10; A61K 31/4439

(52) U.S. Cl. 424/471; 424/485; 424/488; 424/486; 424/487; 424/484; 514/338

(57) ABSTRACT

The present invention relates to an oral drug delivery system for poorly soluble drugs, which can provide sustained near zero order release of poorly water soluble drugs from erodible matrix systems. Erodible matrix core is prepared using at least one active and erosion modulators in a matrix of low molecular weight and high molecular weight hydrophilic polymers in combination with a pH sensitive polymer which enable uniform hydration, controlled erosion and pH independent drug release through out GIT. The core optionally contains solubilizers. The core is optionally coated using combination of low molecular weight water soluble and water insoluble polymers, plasticizer and fillers, which provides for drug release, following a lag time, which also helps to reduce food effects in the stomach.

### FIELD OF THE INVENTION

The present invention relates to drug delivery system for poorly soluble drugs, which can provide sustained near zero order release of poorly water soluble drugs from erodible matrix systems.

### BACKGROUND OF THE INVENTION

An extended (also known herein as “sustained” or controlled) release dosage form of drugs is more desirable than an immediate-release dosage form. Ideally, the controlled release dosage form may provide patients with a convenient dosage regimen that allows less frequent dosing, thus enhancing compliance. Controlled release dosing may also reduce peak-related side effects, maintain therapeutic concentrations throughout the dosing period avoiding periods of insufficient therapeutic plasma concentrations between doses. Within the spectrum of the extended release dosage forms, certain types are more preferred than others.

Drugs having first-order kinetics exhibit an initial high-blood level of the drug followed by an exponential decrease in blood concentration. It has been noted that this kinetic model may be problematic because therapeutic effectiveness will not ensue when blood concentrations of the drug fall below certain levels. Furthermore, some drugs are toxic at high-blood level concentrations, and it is difficult to achieve a balance between effective levels and toxic levels when blood concentrations fall off so rapidly.

A more ideal delivery of drugs would follow zero-order kinetics, wherein blood levels of drugs would remain constant throughout the delivery period. This ideal delivery is particularly important in certain classes of medicines intended, for example, for antibiotic delivery, heart and blood pressure maintenance, pain control, and antidepressants. See, Landgraf et al, Polymer Micro carriers Exhibiting Zero-Order Release, Drug Delivery Technology, http://www.druqdeliverytech.com/cqi-bin/articles.cqi?idArtic le=114 (2005). Accordingly, extended release dosages generally prefer a zero order release profile. In a typical zero order profile, the release of active ingredient would be relatively constant over reasonably extended periods of time.

In prior art osmotic drug delivery systems are used for pH independent controlled delivery of drug substances. U.S. Pat. No. 4,816,263, U.S. Pat. No. 4,946,687, U.S. Pat. No. 4,950,486, . . . U.S. Pat. No. 5,030,456, U.S. Pat. No. 3,845,770, U.S. Pat. No. 3,916,899, U.S. Pat. No. 3,995,601, U.S. Pat. No. 4,008,719, U.S. Pat. No. 4,111,262, U.S. Pat. No. 4,160,020, U.S. Pat. No. 4,327,725 are each related to osmotic dosage forms, and their disclosures are also hereby incorporated by reference in their entirety.

Further U.S. Pat. Nos. 4,765,989, 5,208,037, and 5,019,397 report osmotic release control formulation that exhibit a constant rate of drug release rate following zero order kinetics. However, the manufacturing process of such formulation is very complicated and costly. Additionally, the drug contained therein is not fully bioavailable (John S Grundy and Robert TY. Foster, clin.Pharmacokinet, 30(1): 28-51919960).

Accordingly, there have been numerous efforts to develop a sustained-release formulation which can maintain an effective in vivo drug level for more than 24 hours. For instance, Japanese Patent Publication No. 6001716A (1994. Jan. 11) discloses a solid dispersion formulation based on a hydroxypropyl methylcellulose matrix and European Patent Publication No. 521310A (1993. Jan. 7), a formulation based on a mixture of hydroxypropyl cellulose and hydroxypropyl methylcellulose which is a water-soluble polymer gelation agent. Further, Japanese Patent Publication No. 62077335A (1987. Apr. 9) teaches a gel forming formulation based on carboxyvinyl polymer; Japanese Patent Publication No. 03169814A (1991. Jul. 23), a formulation based on a mixture of a water-soluble polymer such as hydroxypropyl cellulose, hydroxypropyl methylcellulose, polyvinyl pyrrolidone and methylcellulose, and a water insoluble polymer such as microcystal cellulose; and European Patent No. 274176B (1992. May 27), a sustained release formulation based on polyvinyl pyrrolidone. Unfortunately, these formulations have the drawback that a constant rate of drug release cannot be maintained throughout required interval due to the formation of a gel membrane on the outer shell of the formulation, leaving a non-gelated core. It has been reported that a formulation of a drug prepared by using a monoglyceride gel carrier releases the drug at a constant rate that follows zero order kinetics for 24 hours, when brought into contact with a hydrophilic matrix or a water soluble matrix (Korean Patent No. 10-0216624 (1999. May 31)). However, this formulation is of an erosion type and has the problem of easy degradation by contractive movements of the gastrointestinals

### SUMMARY OF THE INVENTION

Accordingly, it is the objective of the present invention to provide novel oral drug delivery system for poorly soluble drugs for oral administration, which upon in vivo administration, is capable of releasing the drug at a constant rate following nearly zero order kinetics through out complete release of poorly soluble drugs, the release rate of drug being affected little by GI motility and over come problems related to osmotic drug delivery system.

In accordance with one aspect of the present invention, there is provided a sustained release composition of poorly soluble drugs for oral administration, comprising an active agent in solubilized form and erosion modulators in a matrix of low molecular weight and high molecular weight hydrophilic polymers in combination with a pH sensitive polymer which enables uniform hydration, controlled erosion and pH independent drug release through out GIT. Optionally the composition of the present invention is further coated with a functional coating comprising combination of low molecular weight water soluble and water insoluble polymers, plasticizer and fillers, which provides for drug release, following a lag time.

### DETAILED DESCRIPTION OF THE INVENTION

The present invention novel oral delivery system for poorly soluble drugs provides a sustained release composition of poorly soluble drugs for oral administration, comprising an active agent in solubilized form and erosion modulators in a matrix of low molecular weight and high molecular weight hydrophilic polymers in combination with a pH sensitive polymer that enables uniform hydration, controlled erosion and pH independent drug release through out GIT. Optionally the composition of the present invention is further coated with a functional coating comprising combination of low molecular weight water soluble and water insoluble polymers, plasticizer and fillers, which provides for drug release, following a lag time.

The inventive drug delivery system for poorly soluble drugs contains at least one active in erodible matrix. The erodible matrix comprising a mixture of low molecular weight and high molecular weight hydrophilic polymers to enables controlled erosion, thereby providing sustained release of active agent.

The erodible matrix additionally comprises of pH sensitive enteric polymer for providing pH independent release of active through out GIT.

The composition of present inventive drug delivery system is further coated with a functional coating comprising combination of pH independent low molecular weight water soluble and water insoluble polymers, plasticizer and fillers, which provides for drug release, following a lag time and reduce food effect in the stomach. Such composition while providing nearly zero order release for poorly soluble drugs provide an advantage over the osmotic drug delivery system due to simple manufacturing technology and reduce manufacturing time.

The drug delivery system for poorly soluble drugs provides an oral drug delivery system comprising a core consists of at least one active in an erodible matrix to provide extended release of the active.

The active agent in said composition could be any one of those drug which are suitable for use in sustained-release drug formulations. Exemplary drugs include but not limited to antihypertensive drugs such as isradipine, nifedipine, doxazocin, amosulralol, felodipine, lercanidipine, lecidipine, nicardipine, fosinopril, imidaprile, clizapril, perindopril, losartan, irvesartan, candesartan. steroidal drugs, antidiabetic drugs gliclazide, glimepirideand glipizide, preferably isradipine and nifedipine. The active agent may be used in the range of about 0.5-60 wt %, preferably about 1 to 30%.

The inventive drug delivery system also comprises water soluble low molecular weight and high molecular weight polymer.

The low molecular weight polymer enables rapid hydration while the high molecular weight polymer provides high gel strength to release drug in controlled manner. The combination of polymer erodes at a constant rate and provides nearly zero order release profile of the drug.

Water soluble low molecular weight and high molecular weight polymer could be selected from among, saccharides, cellulose derivatives, gums, vinyl polymers, acrylates, polyethylene derivatives, etc. and mixtures thereof.

The above hydrophilic polymer used could be but not limited to saccharides, dextrin, polydextrin, dextran, pectin, pectin derivatives, alginate, polygalacturonic acid, xylan, arabinoxylan, arabinogalactan, starch, hydroxypropyl starch, amylose, amylopectin, etc. As for the cellulose derivatives, hydroxypropylmethylcellulose, hydroxypropylcellulose, hydroxymethylcellulose, hydroxyethylcellulose, methylcellulose, sodium carboxymethylcellulose, cellulose acetate, hydroxyethylmethylcellulose, etc. Guar gum, locust bean gum, tragacantha, carrageenan, acacia gum, arabia gum, gellan gum, etc. may be used, for the gums, while for the proteins, gelatin, casein, etc. For the polyvinyl derivatives, polyvinyl alcohol, polyvinylpyrrolidone, polyvinylacetaldiethylaminoacetate, etc. As for the polymethacrylate copolymers, poly(butyl methacrylate, (2-dimethylaminoethyl)methacrylate, methylmethacrylate) copolymer, poly(methacrylic acid, methylmethacrylate) copolymer, poly(methacrylic acid, ethylacrylate) copolymer, etc. While for the polyethylene derivatives, polyethylene glycol, polyethylene oxide, etc. And, for the carboxyvinyl polymers, carbomer, etc. Preferable cellulose ether derivatives and most preferably Hydroxypropylmethylcellulose, is used.

The low molecular weight polymer may be used in the range of about 5 to about 70 wt % and preferably about 10 to about 40 wt %. The high molecular weight polymer may be used in the range of about 5 to about 70 wt % and preferably in the range of 10 to 40 wt %.

The pH sensitive polymer in the inventive drug delivery system could be selected from among enteric polymers. Hydrophilic matrix based tablet has a faster hydration rate in pH 1.2 buffer in comparison with pH 6.8 phosphate buffer (phosphate ions compete for water of hydration), hence shows faster erosion in pH 1.2 buffer, when enteric polymer which insoluble at acidic pH is added to the HPMC matrix of hydrophilic polymer it blocks the pores formed in the matrix ad can give pH independent drug release profile.

Example of such polymers could be but not limited to Cellulose acetate phthalate, Hydroxypropyl methyl cellulose phthalate, Hydroxypropyl methyl cellulose acetate succinate, Methacrylate copolymers, Shellac, Zein, poly vinayl acetate phthalate, more preferably hydroxyl propyl methyl cellulose phthalate and hydroxylpropylmethyl cellulose acetate succinate, most preferably hydroxylpropylmethyl cellulose acetate succinate is used. The pH sensitive enteric polymer can be used in a range from about 0.5 to 30 wt %, preferably about 1 to 10 wt %.

The present invention novel drug delivery system for poorly soluble drugs comprising hydrophilic water soluble and water insoluble fillers as erosion modulators. An appropriate blend of hydrophilic water soluble and water insoluble fillers enables modulation of rate of erosion of matrix.

Water soluble fillers could be used include but are not limited to carbohydrates such as mannitol, sorbitol, arabinose, ribose, xylose, glucose, fructose, mannose, galactose, sucrose, maltose, lactose, raffinose, high molecular weight polyethylene glycols, electrolytes such as sodium chloride, sodium dihydrogen phosphate, sodium and potassium bicarbonates etc. More preferably carbohydrates and its derivatives and most preferably lactose or manitol can be used. In present invention water soluble fillers can be used in range from about 5 to about 75 wt % and preferably about 20 to about 60 wt %.

Water insoluble fillers could be cellulose and its derivatives, calcium carbonate, magnesium carbonates, magnesium oxides, Dicalcium phosphate, starch and its derivatives can be used, more preferably cellulose and its derivatives and most preferably micro crystalline cellulose is used. Water insoluble fillers can be used in range from about 5 to about 75 wt % and preferably about 10 to about 40 wt %.

The present invention novel oral drug delivery system for poorly soluble drugs comprises coating, which enables a lag time before release of active agent. The coat enables matching the profile with osmotic drug delivery system which is know to exhibit lag time.

Example of water soluble polymer in coating could be but not limited to low viscosity grade Methylcellulose, Hydroxypropylmethylcellulose, Hydroxyethylcellulose, hydroxypropylcellulose, Hydroxylethylmethylcellulose, Carboxymethylcellulose and Sodium carboxymethylcellulose, polyvinyl pyrrolidone more preferably cellulose ether derivatives mot preferably low molecular weight hydroxyl propyl methyl cellulose can be used. Water soluble polymers can be used in range from about 20 to about 100 wt %, preferably about 60 to about 100 wt %.

Example of water insoluble polymer in coating could be but not limited to ethyl cellulose and its derivatives, Cellulose acetates and vinyl polymers. More preferably cellulose derivatives and most preferably low molecular weight ethyl cellulose and its derivatives can be used. Water insoluble polymers can be used in a range from about 1 to about 30 wt %, preferably about 1 to about 15 wt %.

Coating may include other additives like titanium dioxide, talc, fillers and plasticizer like dibutyl sebacate, triethylcitrate, poly ethylene glycol derivatives, castor oil etc. The coating could be applied from about 2 to about 15% weight gain, preferably about 3 to about 10% weight gain.

The present invention novel oral drug delivery system for poorly soluble drugs may additionally contain color coating. The color coating can be applied in the range of about 1 to about 10% weight gain preferably about 2 to about 5% weight gains.

In addition to above excipients, matrix may contains solubilizers like sodium lauryl sulfate, vitamin E derivatives, Poloxamers, tween 80, low molecular weight cellulose derivatives, low molecular weight pyrollidone derivatives can be used, preferably sodium lauryl sulphate and poloxamer and most preferably sodium lauryl sulphate can be used.

In addition Lubricants like Talc, magnesium stearate, calcium stearate, zinc stearate, lauryl sulfate, hydrogenated vegetable oil, sodium benzoate, sodium stearyl fumarate, glyceryl monostearate and glidants, antiadherent and other standard tabletting excipients known in art can be used.

The formulation would be designed as a compressed tablet by standard tabletting techniques, and coated using standard coating equipment like coating pans, automatic coater or fluid bed coater.

### BRIEF DESCRIPTION OF DRAWING

FIG. 1 compares the release profile of uncoated isradipine extended release formulation with and without enteric polymer in pH 1.2 buffer and pH 6.8 buffer.

FIG. 2 compares the drug release profile of uncoated and coated isradipine extended release tablet with commercially available osmotic drug delivery system DynaCirc CR.

FIG. 3 compares the release profile of poor water soluble drug isradipine in different pH medium for coated tablet.

### EXAMPLES

This invention will be further described in detail as in the following, yet this invention is not limited by the following examples.

Practical and preferred embodiments of the present invention are illustrated as shown in the following examples. However, it will be appreciated that those skilled in the art may, in consideration of this disclosure, make modifications and improvements within the spirit and scope of the present invention.

### Example 1

### Preparation of Isradipine Tablet with and without Enteric Polymer (10 Mg)

Procedure: Isradipine, Methocel K15M, Methocel K4M, Hydroxy propyl methyl cellulose acetate succinate, Surfactant, lactose and microcrystalline cellulose are passed through # 40. Blend is granulated with binder solution containing PVP K30, granules are dried and milled through 1 mm screen. Granules are lubricated with aerosil 200 and magnesium stearate. Granules ready for compression were compressed using 10 mm standard concave punches at a hardness of 8-10 KP. Formula (A) contains enteric polymer HPMC acetate succinate, where as enteric polymer is absent in case of formulation (B). Composition of the formulation (A) and (B) is given in Table 1

TABLE 1

| | (A) Qty/Tab | (B) Qty/Tab |
|---|---|---|
| | (mg/Tab) | (mg/Tab) |
| | (With enteric | (Without enteric |
| Ingredients | polymer) | polymer) |
| Isradipine | 10.00 | 10.00 |
| PVP K30 | 20.00 | 20.00 |
| Surfactant | 10.00 | 10.00 |
| Methocel K100LV | 40.00 | 40.00 |
| Methocel K4M | 30.00 | 30.00 |
| HPMC acetate succinate | 40.00 | — |
| Microcrystalline Cellulose | 55.00 | 55.00 |
| Mannitol | 189.00 | 229.00 |
| Colloidal silicon dioxide | 2.00 | 2.00 |
| Magnesium stearate | 4.00 | 4.00 |
| Total | 400.00 | 400.00 |

### Example 2

### Preparation of Isradipine Tablet with and without Coating

Isradipine, Methocel K15M, Methocel K4M, enteric polymer, Surfactant, lactose and microcrystalline cellulose are passed through # 40. Blend is granulated with binder solution containing PVP K30, granules are dried and milled through 1 mm screen. Granules are lubricated with aerosil 200 and magnesium stearate. Granules ready for compression were compressed using 10 mm standard concave punches at a hardness of 8-10 KP. Subsequently 10 wt % coating applied to formulation (B) where as (A) is uncoated tablet formulation as shown in Table-II.

TABLE II

| | (A) | (B) |
|---|---|---|
| | Qty/Tab (mg/Tab) | Qty/Tab(mg/Tab) |
| Ingredients | (Uncoated Tablet) | (Coated Tablet) |
| Isradipine | 10.00 | 10.00 |
| PVP K30 | 10.00 | 10.00 |
| Surfactant | 10.00 | 10.00 |
| Methocel K4M | 46.00 | 46.00 |
| Methocel K100LV | 44.00 | 44.00 |
| Enteric polymer | 15.00 | 15.00 |
| Microcrystalline Cellulose | 80.00 | 80.00 |
| Mannitol | 144.00 | 144.00 |
| Colloidal silicon dioxide | 2.00 | 2.00 |
| Magnesium stearate | 4.00 | 4.00 |
| Total | 365.00 | 365.00 |
| Coating (5%) | | |
| HydroxypropylmethylcelluloseE5 | — | 90.00% |
| Ethylcellulose 4 cP | — | 5.00% |
| Triethylcitrate | — | 5.00% |
| Isopropyl Alcohol (80 wt %) | | |
| Purified Water (20 wt %) | | |
| Total | — | 383.00 |

### Testing Example I

### Comparative Dissolution Profile of Uncoated Isradipine Extended Release Formulation With and without Enteric Polymer in Biphasic Medium

Table I shows composition of Isradipine formulation with (A) and without (B) addition of enteric polymer which is insoluble in acidic pH. These tablets have been tested in biphasic medium with 0.2% tween 80 as surfactant. 1 Hr in pH 1.2 buffer and further profile in pH 6.8 buffer has been carried out. USP apparatus-II with 75 RPM was used to measure drug release profile.

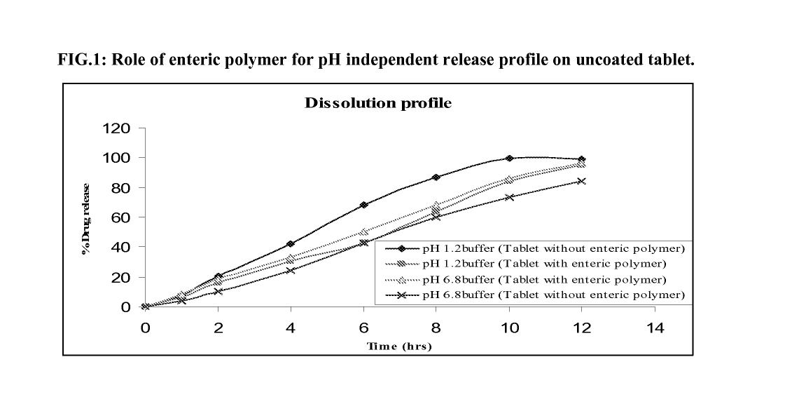

As shown in FIG. 1 tablets without HPMC acetate succinate in pH 1.2 shows faster drug release profile (approx-85% in 8 hrs), where as release profile in pH 6.8 phosphate buffer showed slower release profile (approx-80% in 12 hrs), where as tablets with HPMC acetate succinate showed pH independent release profile. This is because HPMC based matrix tablet has a faster hydration rate in pH 1.2 buffer in comparison with pH 6.8 phosphate buffer (phosphate ions compete for water of hydration), hence faster erosion in pH 1.2 buffer.

When enteric polymer HPMC acetate succinate which is insoluble at acidic pH is added to the HPMC matrix, it blocks the pores formed in the matrix and reduces hydration rate of the matrix, hence reduce erosion of the matrix and can give pH independent drug release profile.

### Testing Example II

### Comparative Dissolution Profile of Coated and Uncoated Formulation with Commercially Available Osmotic Drug Delivery System

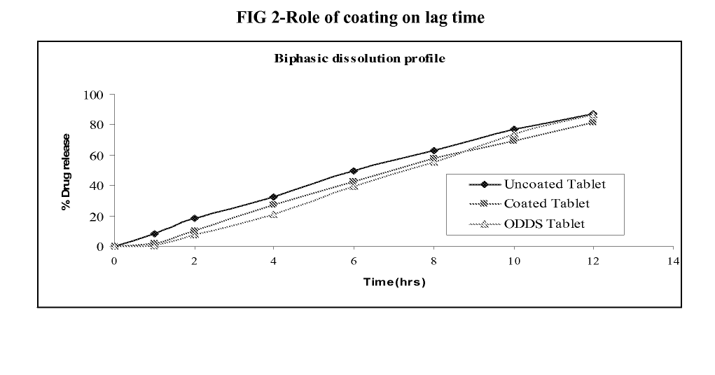

Coated and Uncoated Tablets have been tested in pH 1.2 buffer followed by pH 6.8 buffer with 0.2% tween 80 in USP Apperatus Type-11 at 75 RPM. The result are compared with innovator tablet having osmotic drug delivery system in FIG. 2

As shown in FIG. 2, coated tablet showed delay in drug release for initial 2 hrs followed by constant drug release which is nearly zero order. This delayed release of coated tablet in comparison of uncoated tablet helps to match the profile with innovator drug release profile, which is osmotic drug delivery system. pH independent nature of the coating also ensure less variability for drug release in the stomach in fed and fasted condition. The tablet of the present invention maintains systematic drug release with a single-phase matrix, which simplifies the manufacturing method and process and reduces manufacturing time. And, the extended release tablet of the present invention has a uniform dissolution profile. This invention also overcomes the disadvantage of complicated and costly osmotic drug delivery system. The present invention also release complete drug from the matrix, which is not the case in osmotic drug delivery system. So the present invention can be a good alternative for osmotic drug delivery system.

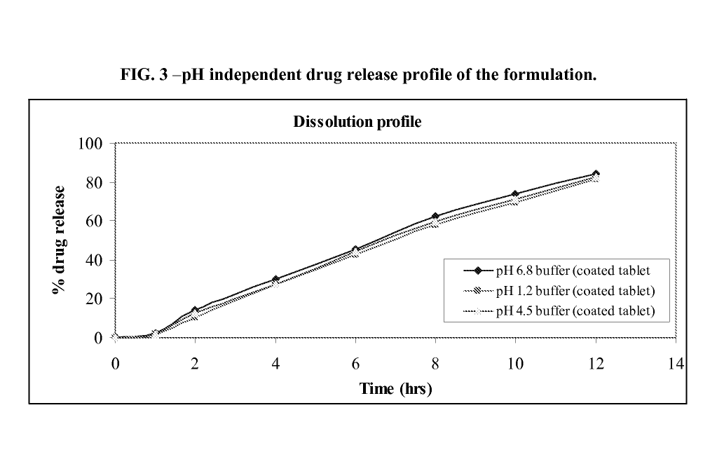

As shown in FIG. 3 coated formulation (B) as per Table-II gives pH independent drug release profile in pH 1.2 buffer, pH 4.5 buffer and pH 6.8 buffer. Similarity factor in release profile at all three different pH is more than 50. This shows that present invention novel oral drug delivery system for poorly soluble drug has potential to provide pH independent controlled release drug profile with a lag time and hence it can be a good alternative to osmotic drug delivery system with a simple manufacturing technology.

As apparent from the above description, the present inventive delivery system offers a good alternative for osmotic drug delivery system.

The present inventive drug delivery system is capable of releasing the drug component slowly at a constant rate in controlled manner. Thus the drug level in blood can be maintained constant over extended period of time with a single administration a day.

The present inventive drug delivery system for gives the initial lag for drug release similar to osmotic drug delivery system and then follow controlled erosion of the matrix providing nearly zero order drug release profile similar to osmotic drug delivery system.

The present inventive drug delivery system is a simple matrix technology compared to complex osmotic drug delivery system, hence it is easy to manufacture, saves cost and time.

Those skilled in art will appreciate that the concept and specific embodiments disclosed in the foregoing description may be readily utilized as a basis for modifying or designing other embodiment for carrying out the purpose of the present invention. Those skilled in art will also appreciate that such equivalent embodiments do not depart from the spirit and scope of present invention as set forth in appended claims.

1. An drug delivery system for poorly soluble drugs comprising a core consisting of at least one active pharmaceutical or active pharmaceutical in solubilized form in an erodible matrix to provide extended controlled release of the active.

2. The drug delivery system as set forth in claim 1, wherein drug constitute from 0.5-60 wt %.

3. The drug delivery system as set forth in claim 1, wherein the active pharmaceutical drug could be but not limited to antihypertensive drugs such as isradipine, nifedipine, doxazocin, amosulralol, felodipine, lercanidipine, lecidipine, nicardipine, fosinopril, imidaprile, clizapril, perindopril, losartan, irvesartan and candesartan. Steroidal drugs, antidiabetic drugs gliclazide, glimepirideand glipizide, preferably isradipine and nifedipine.

4. The drug delivery system as set forth in claim 1, wherein the erodible matrix is a combination of hydrophilic low molecular weight polymer, high molecular weight polymer, at least one enteric polymer, hydrophilic water soluble and water insoluble erosion modulators and standard tabletting excipients known in the art

5. The drug delivery system as set forth in claim 4, wherein hydrophilic low molecular weight polymer and high molecular weight polymer at least two selected from saccharides, cellulose derivatives, gums, vinyl polymers, acrylates, polyethylene derivatives.

6. The oral drug delivery system as set forth in claim 5, wherein at lease two polymers selected from but not limited to saccharides, dextrin, polydextrin, dextran, pectin, pectin derivatives, alginate, polygalacturonic acid, xylan, arabinoxylan, arabinogalactan, starch, hydroxypropyl starch, amylose, amylopectin, etc. as for the cellulose derivatives, hydroxypropylmethylcellulose, hydroxypropylcellulose, hydroxymethylcellulose, hydroxyethylcellulose, methylcellulose, sodium carboxymethylcellulose, cellulose acetate, hydroxyethylmethylcellulose, etc. Guar gum, locust bean gum, tragacantha, carrageenan, acacia gum, arabia gum, gellan gum, etc. may be used, for the gums, while for the proteins, gelatin, casein, etc. for the polyvinyl derivatives, polyvinyl alcohol, polyvinylpyrrolidone, polyvinylacetaldiethylaminoacetate, etc. As for the polymethacrylate copolymers, poly(butyl methacrylate, (2-dimethylaminoethyl)methacrylate, methylmethacrylate) copolymer, poly(methacrylic acid, methylmethacrylate) copolymer, poly(methacrylic acid, ethylacrylate) copolymer, etc. while for the polyethylene derivatives, polyethylene glycol, polyethylene oxide, etc. and, for the carboxyvinyl polymers, carbomer, etc. preferable cellulose ether derivatives and Hydroxypropylmethylcellulose.

7. The drug delivery system as set forth in claim 5, wherein the low molecular weight polymer may be used in the range of about 5 to about 70 wt % and the high molecular weight polymer may be used in the range of about 5 to about 70 wt %.

8. The drug delivery system as set forth in claim 4, wherein the erodible matrix contains at least one enteric polymer, these could be but not limited to cellulose acetate phthalate, hydroxypropyl methyl cellulose phthalate, hydroxypropyl methyl cellulose acetate succinate, methacrylate copolymers, shellac, zein poly vinayl acetate phthalate.

9. The drug delivery system as set forth in claim 8, wherein the polymer constitute in a range from about 0.5 to about 30 wt %.

10. The drug delivery system as set forth in claim 4, wherein the water soluble erosion modulator could be but not limited to carbohydrates such as mannitol, sorbitol, arabinose, ribose, xylose, glucose, fructose, mannose, galactose, sucrose, maltose, lactose, raffinose and polyethylene glycols, electrolytes, sodium dihydrogen phosphate, sodium and potassium bicarbonates.

11. The drug delivery system as set forth in claim 4, wherein water soluble release modulator constitute from about 5 to about 75 wt %.

12. The drug delivery system as set forth in claim 4, wherein the water insoluble release modulators could be but not limited to cellulose and its derivatives, calcium carbonate, magnesium carbonates, magnesium oxides, Dicalcium phosphate, starch and its derivatives

13. The drug delivery system as set forth in claim 4, wherein water insoluble release modulator constitute from about 5 to about 75 wt %.

14. The drug delivery system as set forth in claim 4, wherein the other standard tableting excipient include but not limited to binder like 1-HPC, polyvinyl pyrolidone, low viscosity grade cellulose derivatives, starch and its derivatives, gelatin, gums and mixture thereof.

15. The drug delivery system as set forth in claim 4, wherein the binder ranges from about 0.1 to about 1 Owt %.

16. The drug delivery system as set forth in claim 4, wherein the other standard tableting excipient include but not limited to solubilizers like sodium lauryl sulfate, vitamin E derivatives, Poloxamers, tween 80, low molecular weight cellulose derivatives, low molecular weight pyrollidone derivatives can be used.

17. The drug delivery system for poorly soluble drugs as set forth in claim 4, wherein the surfactant ranges from about 0.1 to about 10 wt %.

18. The drug delivery system as set forth in claim 4, wherein the other standard tableting excipient include lubricants like talc, magnesium stearate, calcium stearate, zinc stearate, lauryl sulfate, hydrogenated vegetable oil, sodium benzoate, sodium stearyl fumarate, glyceryl monostearate and glidants.

19. The drug delivery system as set forth in claim 1, wherein the erodible core has film coating.

20. The drug delivery system as set forth in claim 19, wherein the film coating layer comprises a film forming agent or a combination of film forming agent and suitable plasticizer.

21. The drug delivery system as set forth in claim 20, wherein a film forming agent can be selected from but not limited to low molecular weight water soluble and insoluble polymers.

22. The drug delivery system as set forth in claim 21, wherein water soluble low molecular weight polymers could be but not limited to methylcellulose, hydroxypropylmethylcellulose, hydroxyethylcellulose, hydroxypropylcellulose, hydroxylethylmethylcellulose, carboxymethylcellulose, sodium carboxymethylcellulose, and polyvinyl pyrrolidone.

23. The drug delivery system as set forth in claim 22, wherein water soluble low molecular weight polymer constitute about 20 to about 100 wt % of the coating weight.

24. The drug delivery system as set forth in claim 21, wherein low molecular weight water insoluble polymer could be but not limited to ethyl cellulose and its derivatives, cellulose acetates and vinyl acetate polymers.

25. The drug delivery system as set forth in claim 24, wherein water insoluble low molecular polymers constitute about 1 to about 30 wt % of the coating.

26. The drug delivery system as set forth in claim 20, wherein plastisizer could be but not limited to dibutyl sebacate, triethylcitrate, poly ethylene glycol derivatives, castor oil etc, preferably triethyl citrate in a range of about 1 to 20 wt % of the coating.

27. The drug delivery system as set forth in claim 19, wherein the coating layer has weight of about 2-15 wt % of the tablet weight.

28. The drug delivery system as set forth in claim 19, wherein the erodible core having a film coating may additionally be coated with a color coat.

29. The drug delivery system as set forth in claim 28, wherein the color coat may contain pharmaceutically acceptable colors like ferric oxides, aluminum lakes etc, it may additionally contains fillers like titanium dioxide, talc etc and suitable plasticizer like polyethylent gycol derivatives, triethyl citrate etc.
